(19) United States
(12) Patent Application Publication
(10) Pub. No.: US 2018/0231491 A1
(43) Pub. Date: Aug. 16, 2018

(54) PAPER-BASED NANO BIOSENSOR DEVICE AND METHOD

(71) Applicant: THE ROYAL INSTITUTION FOR THE ADVANCEMENT OF LEARNING/MCGILL UNIVERSITY, MONTREAL (CA)

(72) Inventors: XINYU LIU, VERDUN (CA); XIAO LI, PALO ALTO, CA (US); CHEN ZHAO, MONTREAL (CA); HAO FU, MONTREAL (CA)

(21) Appl. No.: 15/332,657

(22) Filed: Oct. 24, 2016

Related U.S. Application Data
(60) Provisional application No. 62/245,545, filed on Oct. 23, 2015.

(51) Int. Cl. G01N 27/327; C12Q 1/00; H01G 11/36; G01N 33/543

(52) U.S. Cl. CPC G01N 27/3278; C12Q 1/002; H01G 9/042; G01N 33/54393; G01N 33/5438; H01G 11/36

(57) ABSTRACT

A biosensor device comprises a substrate base having a paper body, the substrate base supporting at least a first electrode having a growth nanomaterial surface on a portion thereof adapted to receive a sample fluid, and a contact end configured for connection to measuring equipment. A substrate cover has a paper body, the substrate cover defining a reaction zone in which the paper is exposed on both sides of the paper body, the substrate cover supporting at least a second electrode and at least a third electrode each having a connection end contacting the reaction zone and a contact end configured for connection to the measuring equipment, wherein the reaction zone is superposed on the growth nanomaterial surface during use for wicking of the sample fluid through the reaction zone.

### CROSS-REFERENCE TO RELATED APPLICATION

This application is claims priority from U.S. Provisional Application No. 62/245,545, filed Oct. 23, 2015, the subject matter of which is incorporated herein by reference in its entirety.

### TECHNICAL FIELD

The present application relates to biosensors for assays.

### BACKGROUND OF THE ART

Point-of-care (POC) testing with biosensors highlights a new paradigm of molecular diagnosis where the diagnostic assays and measurements are performed rapidly and easily by non-professional people on arbitrary sites. POC testing may solve challenges in both developing and developed countries. In developing countries, the costs of diagnoses are a major concern, and centralized hospitals, sophisticated clinically assay equipment, and other facilities are not readily accessible. Point-of-care biosensors, if well-developed to meet standard requirements, may allow fast and low-cost diagnosis or pre-diagnosis in resource-limited regions. In developed countries, although the health-care systems are well established, the hospitals and clinics undergo pressure because of the need for diagnoses and regular check-ups. With point-of-care biosensors, users may conduct tests rapidly without regular visits to hospitals or clinics. Moreover, the point-of-care biosensors can be conveniently connected to the wide-spread telecommunication or internet networks in this era of telemedicine. Low-cost, easy-to-use, and performing biosensors are the cornerstones to the paradigm of POC testing.

Throughout recent advancements, electrochemical immunoassays have been an active research topic in biosensor developments, because immunoassays are prevalent in modern diagnostic tests and electrochemical biosensing mechanisms provide sensitive, quantitative and reliable readouts. To really fulfill their promise, biosensors should have performance comparable to or better than commonly used reliable diagnostic tests, such as the enzyme-linked immunosorbent assay (ELISA) used in clinical laboratories. To this end, functional nanomaterials and sensing mechanisms have been introduced to biosensors for immunoassays. Carbon nanotubes and graphene were added to biosensors for their high surface area as first attempts. More recent research reported directly synthesized silver or gold-based nanomaterials on cellulose paper substratem, and introduced other nanoparticles as part of a label. However, the preparation of such nanomaterials typically requires sophisticated equipment or procedures, along with costly materials. Moreover, any immunoassay protocol requires secondary antibodies, labels and may even require amplifying reagents, and consequently involves relatively complex operations and extra costs of reagents. Hence, there is room for improvement for biosensors that are suitably sensitive, with increased convenience and rapidity of use.

### SUMMARY

In accordance with the present disclosure, there is provided a biosensor device comprising: a substrate base having a paper body, the substrate base supporting at least a first electrode having a growth nanomaterial surface on a portion thereof adapted to receive a sample fluid, and a contact end configured for connection to measuring equipment, and a substrate cover having a paper body, the substrate cover defining a reaction zone in which the paper is exposed on both sides of the paper body, the substrate cover supporting at least a second electrode and at least a third electrode each having a connection end contacting the reaction zone and a contact end configured for connection to the measuring equipment, wherein the reaction zone is superposed on the growth nanomaterial surface during use for wicking of the sample fluid through the reaction zone.

Still further in accordance with the present disclosure, the substrate base and the substrate cover concurrently form a single-piece paper body, the substrate base and the substrate cover separated by a fold line for folding the single-piece paper body into superposing the reaction zone with the growth nanomaterial surface.

Still further in accordance with the present disclosure, layering of hydrophobic material on the substrate cover surrounding and delimiting the reaction zone.

Still further in accordance with the present disclosure, the second and the third electrode have a body printed onto the substrate cover.

Still further in accordance with the present disclosure, the second electrode has a cane shape with an arcuate portion thereof being the connection end of the second electrode with the reaction zone.

Still further in accordance with the present disclosure, the first electrode has an own paper body installable onto the substrate base.

Still further in accordance with the present disclosure, a layer of adhesive on the substrate base is for securing the first electrode thereon.

Still further in accordance with the present disclosure, at least one backing strip may be on the layer of adhesive, the at least one backing strip defining a cutout contoured to receive the first electrode therein.

Still further in accordance with the present disclosure, the paper body of the first electrode has a carbon layer extending from the contact end to the growth nanomaterial surface.

Still further in accordance with the present disclosure, the paper body of the first electrode is layered with a hydrophobic material except on the growth nanomaterial surface.

Still further in accordance with the present disclosure, a substrate flap has paper body and a filter membrane superposed onto an exposed paper zone of the paper body, the exposed paper portion superposed onto the growth nanomaterial surface for wicking of a sample liquid filtered by the filter membrane, through the exposed paper zone, and onto the growth nanomaterial surface.

Still further in accordance with the present disclosure, the substrate base and the substrate flap concurrently form a single-piece paper body, the substrate base and the substrate flap separated by a fold line for folding the single-piece paper body into superposing the exposed paper zone with the growth nanomaterial surface.

Still further in accordance with the present disclosure, a substrate flap has a washing element thereon, the substrate base and the substrate flap concurrently form a single-piece paper body, the substrate flap being separable from the substrate base for washing the substrate base.

Still further in accordance with the present disclosure, the growth nanomaterial surface of the first electrode includes zinc oxide nanowires.

Still further in accordance with the present disclosure, at least one capture molecule is covalently bounded to at least one of said first electrode, second electrode and third electrode.

Still further in accordance with the present disclosure, said capture molecule is a probe, an antibody or an antigen.

Still further in accordance with the present disclosure, said capture molecule is specific for cardiac troponin I (cTnI), BNP32, D-Dimer, or a viral protein.

Still further in accordance with the present disclosure, said viral protein is p24.

In accordance with a further embodiment of the present disclosure, there is provided a method for preparing a biosensor device for an assay comprising: exposing a growth nanomaterial surface of a first electrode to a sample fluid; after capturing the sample fluid on the growth nanomaterial surface, applying a paper reaction zone in contact with a second and a third electrode against the growth nanomaterial surface of the first electrode to cause wicking action of the captured sample fluid through the paper reaction zone; adding an electron mediator to the paper reaction zone; and connecting the electrodes to measurement equipment to analyze the sample fluid.

In accordance with the further embodiment, applying the paper reaction zone against the growth nanomaterial surface comprises folding a first substrate portion against a second substrate portion.

Still in accordance with the further embodiment, folding a first substrate portion against a second substrate portion comprises adhering the first substrate portion to the second substrate portion.

Still in accordance with the further embodiment, adhering the first substrate portion to the second substrate portion comprises removing a backing strip from the first substrate portion to expose an adhesive.

Still in accordance with the further embodiment, comprising adhering the first electrode to a substrate base prior to applying the paper reaction zone against the growth nanomaterial surface.

Still in accordance with the further embodiment, exposing the growth nanomaterial surface of the first electrode to the sample fluid comprises deposing the sample fluid on a filter membrane, and wicking part of the sample fluid from the filter membrane through a paper substrate and onto the growth nanomaterial surface.

In accordance with yet another embodiment, there is provided a method of detecting a targeted molecule in a sample fluid comprising: capturing said sample fluid to a biosensor device as defined above, said biosensor device comprising at least one capture molecule covalently bounded to at least one of said first electrode, second electrode and third electrode of the biosensor device specific for said targeted molecule; connecting the electrodes to measurement equipment to analyze the sample fluid detecting the presence of said targeted molecule.

Still in accordance with the further embodiment, washing the electrodes is performed to remove non-specific binding of the targeted molecule to said capture molecule.

Still in accordance with the further embodiment, said capture molecule is a probe, an antibody or an antigen.

Still in accordance with the further embodiment, said capture molecule is specific for cardiac troponin I (cTnI), BNP32, D-Dimer, or a viral protein.

Still in accordance with the further embodiment said viral protein is p24.

### DESCRIPTION OF THE DRAWINGS

FIG. 1 depicts views of a paper-based nano biosensor device in accordance with the present disclosure;

FIG. 2 are views of a set of four of the paper-based nano biosensor device of FIG. 1;

FIG. 3 is a series of sequential views showing a use of the set of FIG. 2 of the paper-based nano biosensor device;

FIG. 4 are views of a set of three of the paper-based nano biosensor device of FIG. 1, for instance as used for testing blood samples;

FIG. 5 is a series of sequential views showing a use of the set of FIG. 4 of the paper-based nano biosensor device;

FIG. 6 are an exemplary views depicts zibc oxide (ZnO)-nanowire growth on a paper-based electrode, with (a) scanning electron microscopy (SEM) image of pristine carbon-ink electrode, (b) SEM image of electrode after ZnO-nanowire growth, (c) SEM image of the cross-section view of a ZnO-nanowire electrode on top of a paper reaction zone, and (d) transmission electron microscopy (TEM) image of the lattice structure of a single ZnO nanowire;

FIG. 7 depicts the X-ray powder diffraction (XRD) spectrum of pristine carbon-ink electrode and ZnO-nanowire electrode;

FIG. 8 depicts biofunctionalization and blocking of the ZnO-nanowire electrode, with (a) illustration of the surface chemistry of biofunctionalization process and (b) XRD spectra of ZnO-nanowire electrodes after different steps of biofunctionalization, including overall survey and close look at the key elements (N1s, O1s, Si2p, C1s, and Zn2p);

FIG. 9 depicts fluorescence measurement results of ZnO-nanowire working electrode after protein immobilization and two subsequent washing steps, inset being a fluorescence image of ZnO-nanowire working electrode after the second washing, with background fluorescence intensity of the pristine ZnO-nanowire electrode subtracted from all the intensity readouts (N=5);

FIG. 10 depicts fluorescence measurement results of blocked ZnO-nanowire working electrode through addition of extra FITC tagged anti-rabbit IgG and subsequent washing, with background fluorescence intensity of the pristine ZnO-nanowire electrode subtracted from all the intensity readouts (N=5);

FIG. 11 depicts an electrochemical characterization of the biosensor device of FIG. 1, with (a) CV measurement results of varied working electrode conditions, (b) bepresentative Nyquist plots obtained with varied working electrode conditions through electrochemical impedance spectroscopy (EIS) measurements in the frequency range of 100 k-0.1 Hz, the inset being the Nyquist plot obtained with blocked working electrode in the frequency range of 20 k-20 Hz, and for both (a) and (b), working electrode #1 to #4 denote bare-carbon, ZnO-nanowire, anti-rabbit-IgG-immobilized, and blocked working electrodes, respectively, and with (c) schematic diagram of the EIS biosensing principle and the equivalent circuit model used for interpret the measured Nyquist plots.

FIG. 12 depicts detection of rabbit IgG in PBS, with (a) representative Nyquist plots of complete biosensor devices of the present disclosure in response to PBS samples with increased rabbit IgG concentrations, and (b) calibration results of rabbit IgG in PBS, with data fitted into an S-curve;

FIG. 13 depicts detection of human immunodeficiency virus (HIV) p24 antigen in human serum, with (a) test results of biosensor devices of the present disclosure with human serum samples with and without p24 antigen, after different incubation times, and with (b) calibration results of biosensor devices of the present disclosure for p24 antigen spiked in HIV-negative human serum samples;

FIG. 14 depicts device stability testing during storage, groups A, B and C include biosensor devices of the present disclosure prepared and stored under different conditions, to test 0, 10 pg/mL, 10 μg/mL p24 antigen samples in human serum samples after storage periods of 0, 2, 4, and 8 weeks;

FIG. 15 depicts resistance results of cross-talk investigation for HIV p24 antigen detection in serum, the biomarker compositions of the groups being: 1 ng/mL HIV p24 antigen and 1 ng/mL hepatitis C virus (HCV) antigen for Group A; 1 ng/mL HIV p24 antigen and 10 μg/mL HCV antigen for Group B; 1 ng/mL HIV p24 antigen only for group C;

FIG. 16 depicts calibration results of three cardiac biomarkers (cardiac troponin I or cTnI, D-dimer, and B-type natriuretic peptide 32 or BNP-32) in artificial blood plasma using the set of biosensor devices of FIG. 4;

FIG. 17 depicts calibration results of standard ELISA tests of the three cardiac markers;

FIG. 18 depicts calibration curves of cTnI and D-dimer in spiked human blood using the set of biosensor devices of FIG. 4; and

FIG. 19 is a schematic view of the biosensor of FIG. 1, with exemplary dimensions.

### DETAILED DESCRIPTION

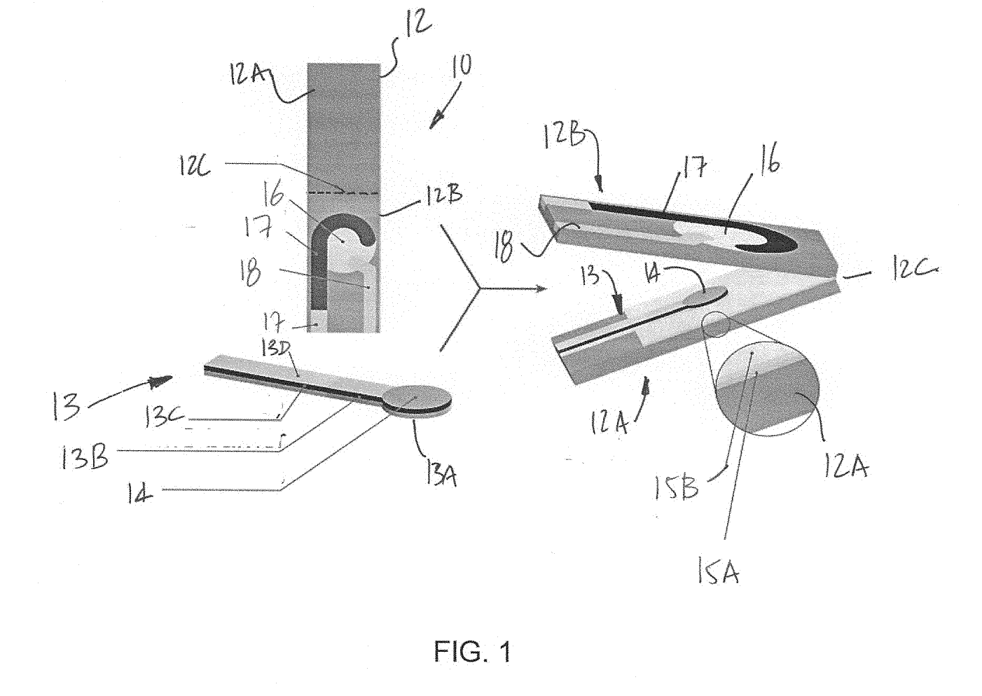

Referring to the drawings and more particularly to FIG. 1, a paper-based nano biosensor device in accordance with the present disclosure is generally shown at 10. For simplicity, the paper-based nano biosensor device is referred to as device 10 or biosensor device 10. The substrate 12 of the biosensor device 10 is made of one or more paper-based strips (a.k.a., paper panels, paper cutouts, paper bodies). The substrate 12 of FIG. 1 is defined by a substrate base 12A and a substrate cover 12B, separated by a fold line 12C. The substrate base 12A and the substrate cover 12B may consist of one integral paper-based strip foldable at fold line 12C, or two distinct strips interconnected by a tape or adhesive as described hereinafter.

A layer of a hydrophobic material (such as hydrophobic wax, hydrophobic photoresist or other hydrophobic polymer) may be used to pattern the surfaces of the substrate 12, by penetrating the thickness of the substrate 12 in pre-defined areas, with hydrophilic patterned reaction zones of the substrate 12 being defined through the hydrophobic material wicked through the substrate 12. The reaction zones will wick fluids.

The substrate base 12A supports a working electrode 13. According to an embodiment, the working electrode 13 is a carbon-printed working electrode that is separate from the substrate base 12A and is secured thereon. Alternatively, the working electrode 13 may be integral with the substrate base 12A. The working electrode 13 may include a paper substrate 13A supporting a carbon-printed layer 13C, a hydrophobic barrier 13B separating a contact end 13D of the working electrode 13 from a surface 14 of nanomaterials. According to an embodiment, the hydrophobic barrier 13B is polydimethylsiloxane (PDMS) although other hydrophobic materials may be used as well. The contact end 13D may include a layer of conductive material, such as silver/silver chloride (Ag/AgCl).

The surface 14 of nanomaterials is provided at an end of the working electrode 13, in conductive contact with the working electrode 13. The nanomaterials for the surface 14 may include any of the following: ZnO nanowires, gold nanostructures (nanowires, nanorods, nanoslices, and nanoflowers) if growth conditions are compatible with paper substrates, graphene and other 2D nanomaterials (e.g., MoS₂) pre-synthesized and added to the working electrode 13, silicon nanowires and carbon nanotubes (CNT) pre-synthesized and added to the working electrode 13, or other growth nanomaterials. ZnO nanowires may be directly synthesized through a cost-effective hydrothermal process.

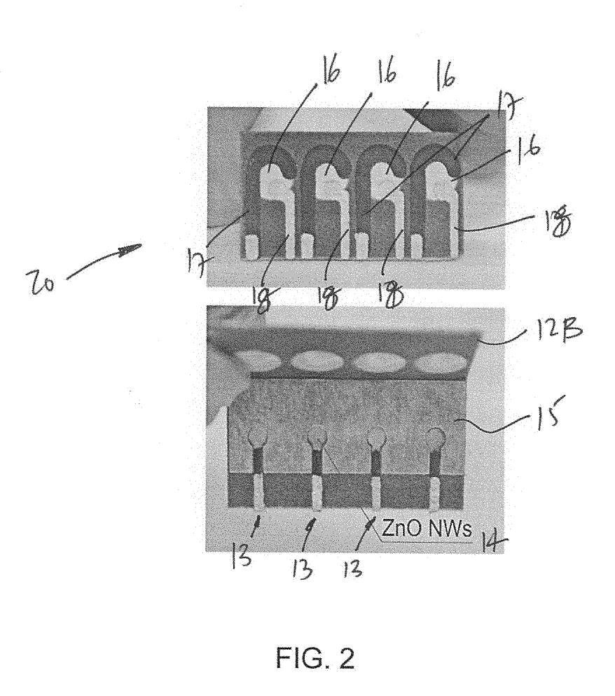

The surface 14 may be surrounded by a layer of adhesive 15A. According to an embodiment in which the working electrode 13 is separate from the substrate base 12A, the layer of adhesive 15A also extends under the working electrode 13 for the working electrode 13 to be adhered to the substrate base 12A. The layer of adhesive 15A may be covered by a removable backing strip 15B to facilitate the manipulation of the substrate base 12A prior to the assay, and to avoid contaminating and affecting the tackiness of the adhesive 15A. As observed in FIG. 2, the backing strip 15B is shaped to contour the surface 14 and so as not to cover the surface 14. The layer of adhesive 15A is used to hold the substrate base 12A to the substrate cover 12B during the assay. Another removable backing strip (having the same outline as the working electrode 13) may also cover the portion of the adhesive layer 15A shaped as the working electrode 13, in the embodiment in which the working electrode 13 is separate from the substrate base 12A. In such a case, the first backing strip is removed to bond the working electrode 13 to the substrate base 12A, and the second backing strip 15B is removed thereafter to hold the substrate base 12A to the substrate cover 12B during the assay. The layer of adhesive 15A may also be used to interconnect the substrate base 12A to the substrate cover 12B at the fold line 12C if they are separate pieces.

The substrate cover 12B supports various components on a side opposite that of the working electrode 13. A reaction zone 16 is defined in the substrate cover 12B. The reaction zone 16 may be a portion of the paper substrate cover 12B that is not covered by the hydrophobic layer, leaving the paper of the substrate 12 exposed, and therefore the reaction zone 16 is essentially a window of exposed paper, on both sides of the paper body. Hence, the reaction zone 16 is without hydrophobic coverage on both sides, for the reaction zone 16 to wick fluids. The reaction zone 16 is positioned in the substrate cover 12B so as to be in register with the surface 14 of nanomaterials when folded about the fold line 12C. Counter electrode 17 and reference electrode 18 extend from an edge of the substrate cover 12B to the reaction zone 16 so as to be in contact with the reaction zone 16, the latter being in contact with the surface 14 of nanomaterials and the biofluids thereon. In the illustrated embodiment, the counter electrode 17 is cane-shaped. The cane-shaped electrode includes a semi-circular carbon electrode portion, and a straight contact end portion. The straight contact end portion is for connecting the electrical signal from the semi-circular electrode portion to measuring equipment. The design rationale of the semi-circular portion is for the counter electrode 17 to have approximately the same area as the working electrode 13 on the substrate base 12. The semi-circular shape also ensures that the inner edge of the counter electrode 17 and the edge of the working electrode 13 have a uniform distance. Other shapes are also considered as well.

The working electrode 13 is an important functional component of the device 10. According to an embodiment, the working electrode 13 is separate from the substrate 12 which contains the reaction zone, the counter electrode 17 and the reference electrode 18. The working electrode 13 needs to go through the growth of the surface 14 of nanomaterials and biofunctionalization, which may involve heating and immersion in solutions. In case of immersion, if the working electrode 13 were directly on substrate 12, the hydrophobicity of the various hydrophobic layers could compromised during the process. If the working electrode 13 is a separate piece adhered to the substrate base 12A after immersion, as described above, the shape of the working end 13 may be readily confined without the need for sophisticated material patterning equipment like inkjet printer, and without the constraint of matching the shape of any reaction zone after patterning the counter electrode 17 and reference electrode 18. Hence, as the separate working electrode 13 is placed underneath the reaction zone 16 such that the surface 14 with nanomaterial faces contacts the reaction zone 16, the functionalized nanomaterial may have close contact with solution or sample liquid contained in the reaction zone 16, enabling suitable electrochemical performance.

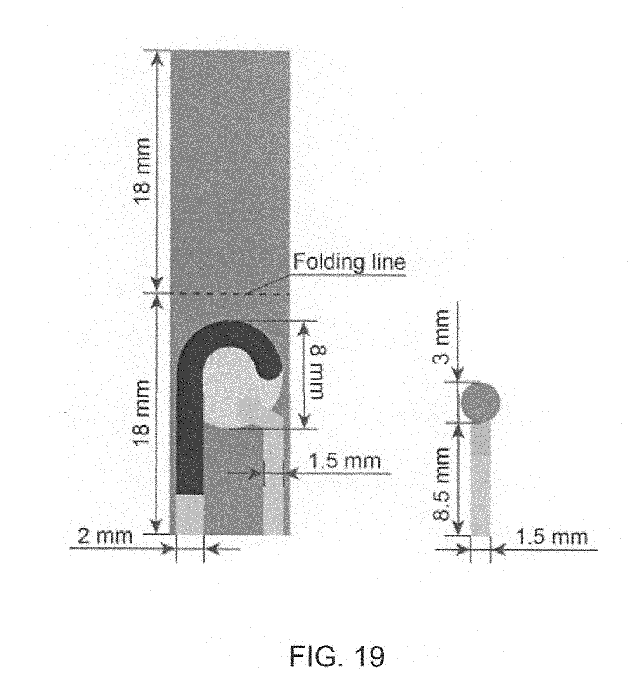

FIG. 19 illustrates exemplary dimensions for the biosensor device 10. The biosensor device 10 may have other dimensions and yet be operational. As an example, it is contemplated to provide the biosensor device with a 15% tolerance on the dimensions shown in FIG. 19.

Referring to FIG. 2, a set 20 of four devices 10 is shown, and is constituted of a single piece of paper substrate 12 (or a single piece of substrate base 12A and single piece of substrate cover 12B) supporting the four devices 10. The set 20 may include two or more of the devices 10. The set 20 may allow many tests to be performed simultaneously, such as four tests in the example of FIG. 2. To facilitate manipulation, a single backing strip 15B may be common to all devices 10 for removal thereof in a single step.

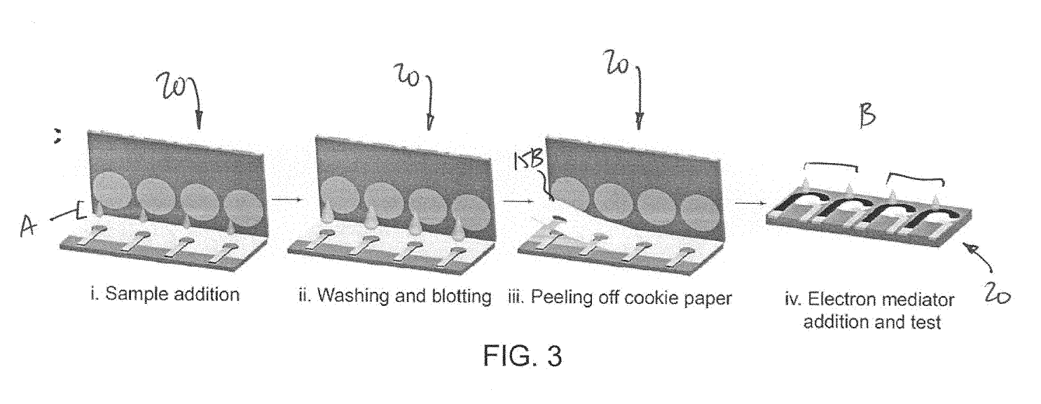

As illustrated in FIG. 3, a method of using the device 10 or the set 20 of devices 10 may include the following steps. According to i), a sample solution A is first introduced on the top of the working electrode(s) 13, for incubation. Alternatively, the working electrode(s) 13 may be immersed in a solution and then adhered to the substrate base 12A. According to ii), washing and blotting steps may then be conducted after incubation, the addition of A, immersing and/or washing and blotting being referred to as biofunctionalizing of the working electrode 13. The direct operations on the surface of the working electrode 13 make the immunological recognition more efficient and the elimination of non-specific binding more thorough. After blotting, the backing strip 15B covering the adhesive layer 15A is removed to expose the adhesive, as in iii. The device 10 or the set 20 of devices 10 is then folded and held together by the adhesive 15A. After addition of an electron mediator solution B through the reaction zone 16, EIS measurement is performed.

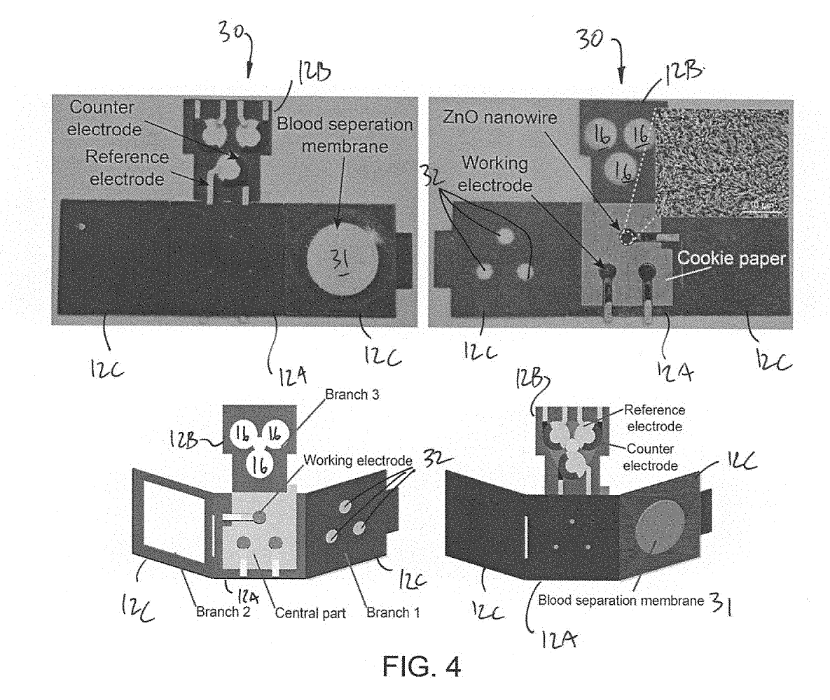

Referring to FIG. 4, another configuration of the device 10 is shown in set 30, and is well suited for immunoassays of real blood samples. Accordingly, like elements will bear like reference numerals. The set 30 distinguishes from the set 20 by the fact that the working electrodes 13 are not all parallel to one another. If the set 30 is used with real blood samples, it conveniently has three working electrodes 13 for measuring the three cardiac biomarkers (cardiac troponin I, BNP32, and D-dimer) used for screening of cardiovascular diseases on patients visiting emergence room (ER) with chest pains. Moreover, the set 30 has a pair of substrate flaps 12C pivotally connected to the substrate base 12A. In an embodiment, the substrate cover 12B and the substrate flaps 12C are connected to the substrate base 12A, so as to be foldable during the use of the set 30 to bring different features on these branches into contact with the three working electrodes 13. The substrate flap 12C has a blood separation membrane 31, to prevent interference of the blood cells with the working electrodes 13. The blood separation membrane 31 may be a commercial blood separation membrane, allowing the biosensor devices 10 in the set 30 to handle whole blood samples to isolate plasma after blood separation. The other substrate flap 12C has a paper absorption pad to blot the washing buffer after the washing step.

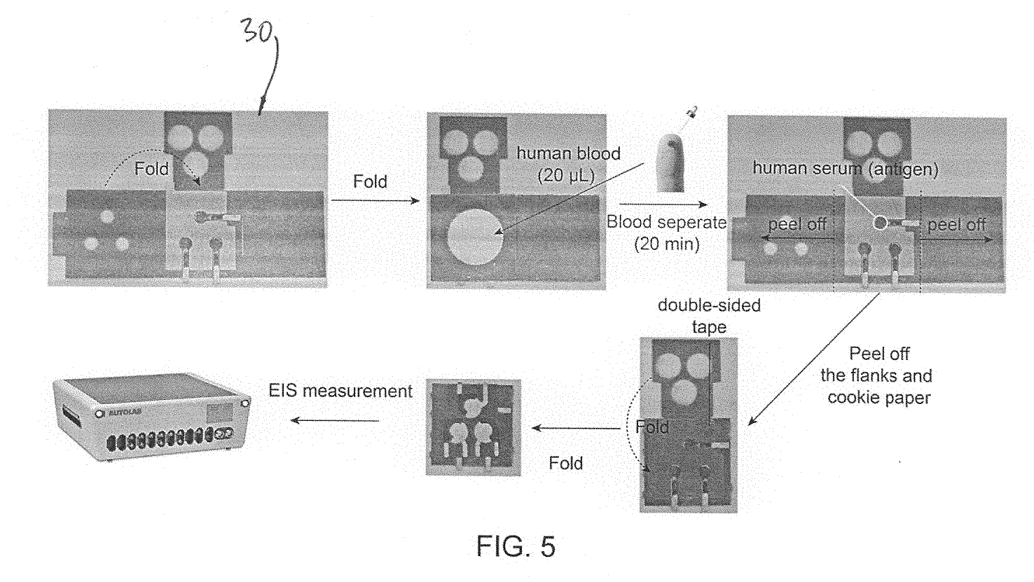

As shown in FIG. 5, the operation procedure of the set 30 of biosensor devices 10 is shown. In the first step of an assay, the substrate flap 12C with three patterned circuit paper inlets 32 (exposed paper on both sides of the flap 12C for instance patterned by hydrophobic layering) and the membrane 31 attached to the three inlets 32, is folded to attach the three inlets 32 to the surfaces 14 of the working electrodes 13. Upon addition of a whole blood sample (20 μl), blood cells are filtered out by the membrane 31, and the remaining plasma gets to the working electrodes 13 through the three inlets 32. The substrate flap 12C with the inlets 32 is then unfolded and torn off the device. Washing buffer is added to each of the working electrodes 13, and blotted away by a blotting paper attached to the other substrate flap 12C that has a piece of paper absorption pad to absorb the extra blocking buffer. The other substrate flap 12C is then torn off as well. Eventually, the substrate cover 12B, which includes counter and reference electrodes 17 and 18 and the reaction zones 16, is folded and attached to contact the working electrodes 13. Electron mediator is added to the reaction zones 16, and EIS measurements are subsequently concocted on the three devices 10. Because the working electrodes 13 are treated with different capture antibodies (specific to the three target biomarkers) during preparation, a specific cardiac protein marker can be detected with each capture antibodies. As such, the biosensor device 10 may be used for detecting three biomarkers in a single run.

For the fabrication of the device 10 as in FIGS. 1 to 4, the reaction zone 16 may be patterned on the substrate cover 12B via wax printing. According to an exemplary embodiment, wax was first printed on a piece of chromatography paper with a commercial printer (ColorQube 8570, Xerox), and heated on a hot plate at 150° C. to form hydrophilic paper reaction zones confined by hydrophobic wax barriers. The counter electrode 17 and the reference electrode 18 was then patterned on top of the reaction zone via stencil printing of carbon ink (E3456, Ercon) and Ag/AgCl ink (E2414, Ercon), respectively. An Ag/AgCl strip was printed to connect the counter electrode 17 to enhance the electrical conductivity between the counter electrode and the measurement equipment. Inks were dried on a hot plate at 65° C. for 20 min after each step of stencil printing. Another piece of chromatography paper was cut into the shape of the working electrode layer using a CO₂ laser cutter (VLS 2.30, Universal Laser Systems), and carbon ink was printed on the circular side (1.5 mm in radius) of the working electrode layer. After the ink was dried, PDMS (mixed with curing agent at w/w ratio of 10:1) was added to both surfaces of the counter electrode layer to form hydrophobic barriers and confine liquids within the circular working electrode area during ZnO nanowire growth and diagnostic assays. An Ag/AgCl strip was patterned on the paper beam of the working electrode layer for electrical connection. The circular working electrode area was then grown with ZnO nanowires through a hydrothermal process, and the ZnO nanowires were subsequently biofunctionalized and blocked as described in the following sections.

### Biosensing Operations

As an example of an experimental biosensing operation, the following actions were performed. A 3 μL of sample solution was added to each functionalized and blocked working electrode 13 of a set 20. After 15-min incubation, the working electrodes 13 were washed with 8 μL of 0.05% Tween®-20 solution. The washing buffer was then blotted away. The set 20 was then folded for the reaction zones 16 to sandwich the working electrodes 13, and 4 μL of 5 Mm K₃[Fe(CN)₆]/K4[Fe(CN)₆] in 100 mM KCl solution were added to each reaction zone 16 as electron mediator. EIS measurement was then performed using a benchtop potentiostat (Autolab PGSTAT302N, Metrohm). As characterization tools, cyclic voltammetry measurements were also performed using the potentiostat.

### Growth of ZnO Nanowires on Paper-Based Electrode 13

The procedure using the devices 10 was modified from the method of growing ZnO nanowires on cellulose paper substrates. The growth of ZnO nanowires involves two steps: seeding and hydrothermal growth. Briefly, ZnO nanoparticle (NP) colloidal ethanol solution was synthesized, with zinc acetate dehydrate and sodium hydroxide solutions. The ZnO NPs were used as the seeds to support ZnO-nanowire growth in the following steps. To seed the nanoparticles in the circular working electrode area, the paper beams of the working electrode layers (coated with carbon ink and PDMS barrier) were clapped between two 1 mm thick glass slides and placed on a 100° C. hot plate, with the circular working electrode areas exposed. 3 μL ZnO NP solution was then added to the surface 14, and let it dry for 3 min. The solution addition and drying were repeated 4 times. The hydrophobic barriers 13B on both sides of the paper beams constrained liquid in the surface 14, to ensure the seeding and hydrothermal growth happened only in the surfaces 14. After seeding, 25 mL of aqueous growth solution, with 50 mM zinc nitrate hexahydrate, 25 mM hexamethylenetetramine, and 0.372 M ammonium hydroxide, was prepared in a 25 mL glass vial, and two 4-working electrode arrays were immersed into the growth solution of one vial. The vial was then sealed and heated in an 86° C. oven for 8 hours to allow the ZnO-nanowire growth. The ZnO-nanowire working electrodes were thoroughly washed with DI water and ethanol, and dried at 86° C., after growth.

### Biofunctionalization and Blocking of the ZnO-Nanowire Working Electrodes 13

A surface chemistry process was used to covalently bind the probe proteins on the surface of ZnO nanowires. First, the ZnO nanowire working electrodes 13 were treated by an air-plasma gun, to introduce —OH groups on the surface 14. Two arrays of four interconnected ZnO-nanowire working electrodes 13 were then immersed into 25 mL 10 mM (3-Aminopropyl)trimethoxysilane (APTMS) ethanol solution in a sealed vial, and heated in a 80° C. oven. After 2-hour heating, the ZnO-nanowire working electrodes 13 were thoroughly washed in ethanol to remove extra APTMS, and dried at 80° C. Then 3 μL 2.5% (v/v) glutaraldehyde (GA) was added to each ZnO-nanowire working electrodes 13 by pipette. After drying in fume hood at room temperature for 30 min, another 3 μL GA at the same concentration was added and dried for another 30 min. Following GA addition, the ZnO-nanowire working electrodes 13 were thoroughly washed in DI water, and dried at 80° C. 3 μL of probe protein solution at a certain concentration (which depends on the target biomarker and sensing range) was then added to each ZnO-nanowire working electrode 13 by pipetting. The incubation was conducted in a 4° C. fridge for overnight. Then, 8 μL of PBS was added to each ZnO-nanowire working electrode 13 by pipetting and blotted to remove extra proteins, as the ending step of the biofunctionalization. The biofunctionalized ZnO-nanowire working electrodes 13 were stored in a 4° C. fridge, if not used immediately.

In order to eliminate non-specific binding, the ZnO-nanowire working electrodes 13 were blocked after biofunctionalization. 3 μL of commercial blocking reagent (Roche) was added to each ZnO-nanowire working electrode 13, and dried at room temperature for 15 min. The addition was repeated twice. Each of the ZnO-nanowire working electrode 13 was then washed by 8 μL of 0.05% Tween 20 PBS solution, and blotted. After dried at room temperature, the ZnO-nanowire working electrodes 13 are assembled into the set 20, to store or perform biosensing experiments.

### Addition of Commercial Stabilizer to the ZnO-Nanowire Working Electrodes 13

The ZnO-nanowire working electrodes 13 were first functionalized with capture antibody, and blocked using the blocking reagent. Extra liquid was removed but the working electrodes 13 were kept from fully dried. The stabilizer solution was prepared by adding 0.01% (v/v) surfactant Tween® 20 solution to stabilizer (StabilCoat, SurModics). 3 μL of the prepared stabilizer solution was dropped to each surface 14, and the working electrode was incubated for 15 min at room temperature before the liquid was blotted away. The working electrodes 13 were then heated on 30° C. hot plate for 1 hour for thorough drying.

### Results

### Design and Operation of the Biosensor Devices 10

### Growth and Characterization of ZnO Nanowires

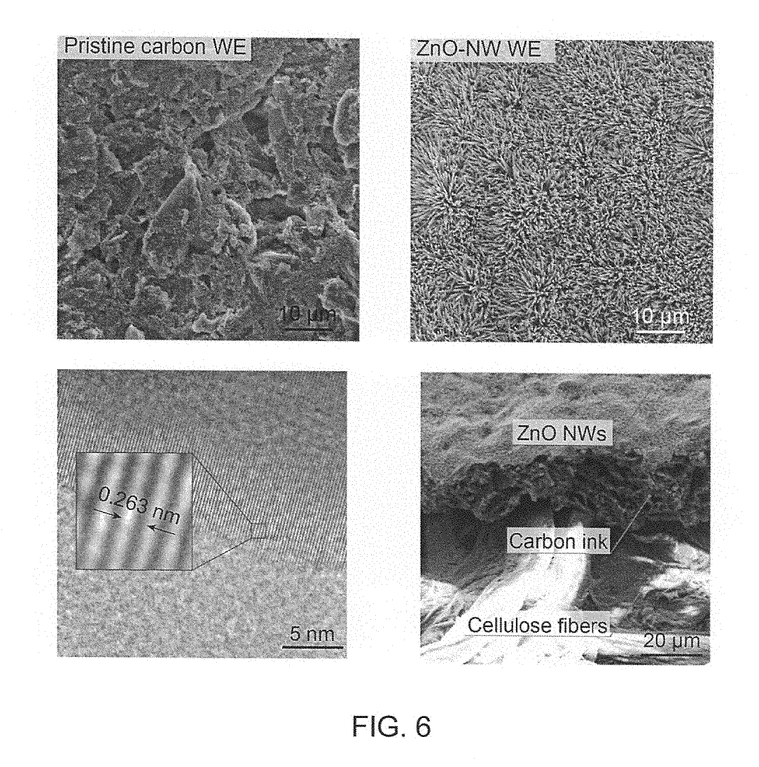

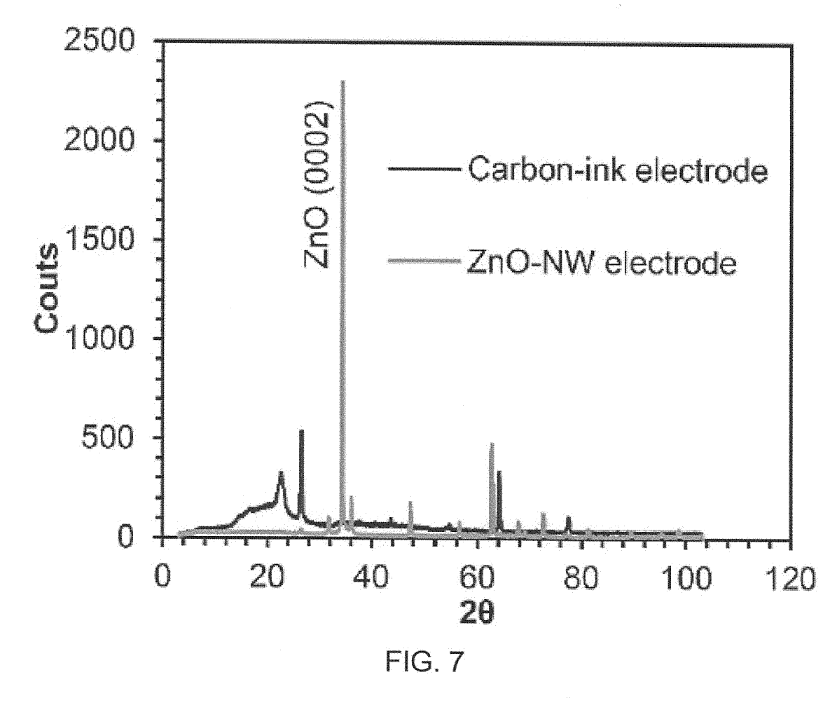

FIG. 6 shows the SEM images of the surfaces 14 of the working electrodes 13, with pristine carbon ink (FIG. 6a) and with ZnO nanowires grown on carbon ink (FIGS. 6b and 6c). Carbon ink formed plate-like non-smooth surface on cellulose fiber after printing (FIG. 6a). Subsequent ZnO nanowire growth yielded high-density ZnO nanowires on the surface of the carbon layer (FIG. 6b). Because the carbon layer is non-smooth, the ZnO nanowires were not uniformly aligned. FIG. 6c shows the SEM image of the cross-sectional view of a ZnO-nanowire-coated working electrode 13. Carbon ink formed a layer about 30 μm thick on the top of cellulose fibers, and ZnO nanowires densely cover the top surface of carbon-ink layer. The SEM imaging revealed that the obtained ZnO nanowires have an average width of 291.47±93.33 nm, an average length of 4.37±0.64 μm, average density of 4.57±0.90/μm2 (N=50, for all the measurements). Although the crystal units of ZnO nanowires are hexagonal and its width may vary along its length, a simple cylindrical shape of individual ZnO nanowires could be assumred and the surface areas ZnO nanowires on top surface the carbon-ink layer could hence be estimated. This calculation implied that the surface area has been increased by a factor of 18.6 with ZnO nanowires, compared to the top surface of carbon-ink layer. The obtained ZnO nanowires were also characterized by transmission electron microscopy (TEM) imaging (FIG. 6d), in which the obtained ZnO nanowires have clear single crystal lattice structures, with lattice spacing of 0.263 nm between (0002) planes. X-ray power diffraction (XRD) on top of pristine carbon-ink working electrode and ZnO nanowires carbon-ink working electrodes showed clear peak changes (FIG. 7). Particularly, ZnO-nanowire carbon-ink working electrode has drastic peak at 2θ=34.44°, corresponding to (0002) planes.

### Biofunctionalization and Blocking of ZnO Nanowires

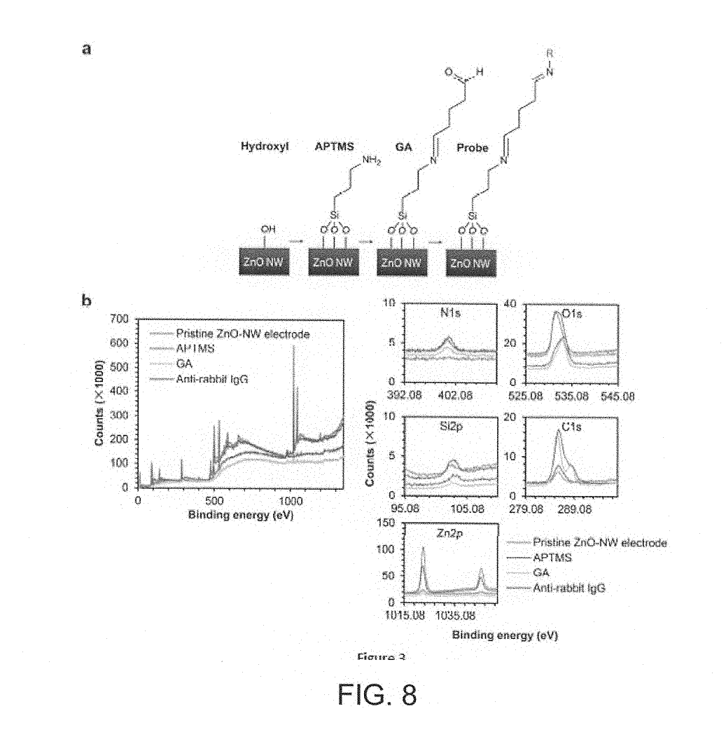

A biofunctionalization process was employed based on covalent bonding, because it is well-accepted that covalent bonding has better stability and uniformity, compared to physisorption or ionic bonding. Specifically, (3-Aminopropyl)trimethoxysilane (APTMS) and glutaraldehyde (GA) were chooses, two commonly used chemicals to covalently bond with amino groups on the protein probes. The entire biofunctionalization process is illustrated in FIG. 8a.

A series of characterizations was performed to examine the results of biofunctionalization. X-ray photoelectron spectroscopy (XPS) was used to analyze the surface chemistries through biofunctionalization, after preparation of pristine ZnO-nanowire electrodes 14, APTMS treatment, GA treatment, and anti-rabbit immunoglobulin G (IgG) immobilization. Before XPS characterization, each working electrode 13 was washed with PBS. Changes of the spectrum can be identified (FIG. 8b). The changes in the spectrum of key elements were consistent with the surface chemistry process. Nitrogen (N1s) had no peak with pristine ZnO-nanowire electrode 13, and was introduced with APTMS, GA and rabbit-IgG which contain NH2 group. Oxygen (O1s) showed a peak that became asymmetric over the introduction of GA and anti-rabbit IgG, because the chemicals contain double bonding of oxygen. Pristine ZnO-nanowire electrodes 13 had slight peak of silicone (Si2p), probably due to the attachment of dusts. The Si2p peak increased with APTMS which has Si atom, and decreased with the following two chemicals. Carbon (C1s) peaks carried more information. With pristine ZnO-nanowire electrodes which has carbon-ink layer and APTMS which has single C—C bonds, there is only one symmetric peak. With GA and anti-rabbit IgG which contain various bonds involving C (such as C═N and COH), the spectrum transformed and implied more than one peak. As for zinc (Zn2p), the peaks decreased with more chemicals introduced to ZnO-nanowire surfaces 14.

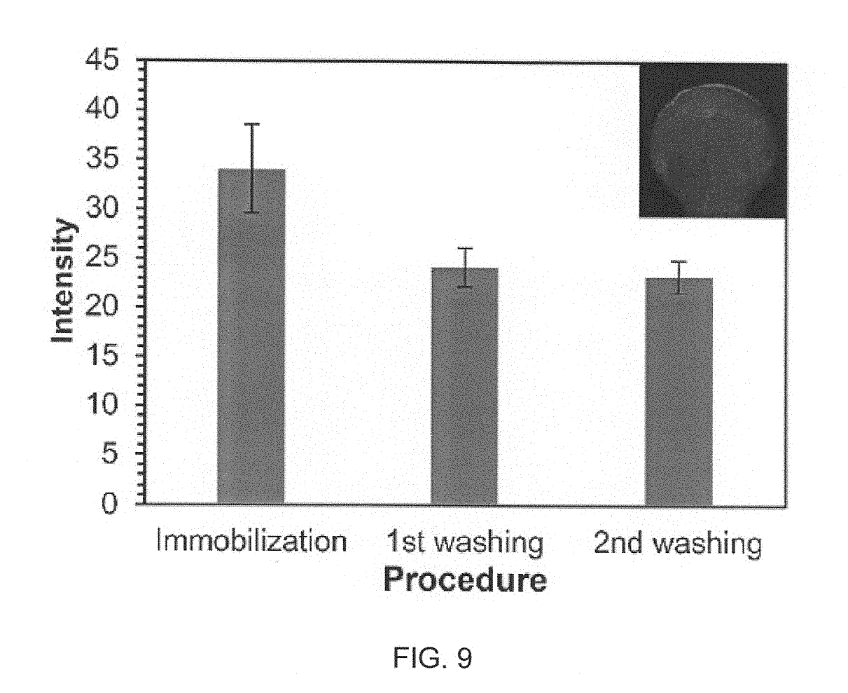

In another experiment, fluorescein isothiocyanate (FITC) tagged anti-rabbit IgG was used as the probe protein immobilized on ZnO-nanowire working electrodes 13, and the working electrodes 13 were examined under fluorescence microscope. The fluorescent anti-rabbit IgG at original concentration (1 mg/mL) was used, in a volume of 3 μL for each working electrode 13. The results are shown in FIG. 9. The fluorescence intensity was decreased by a small portion after the first washing, and the fluorescence intensity was unchanged through the second washing. The results have multiple implications: i) the working electrode 13 stably holds probe proteins after the biofunctionalization process, as the fluorescence intensity remained the same over the second washing; ii) the washing step is effective to remove extra proteins.

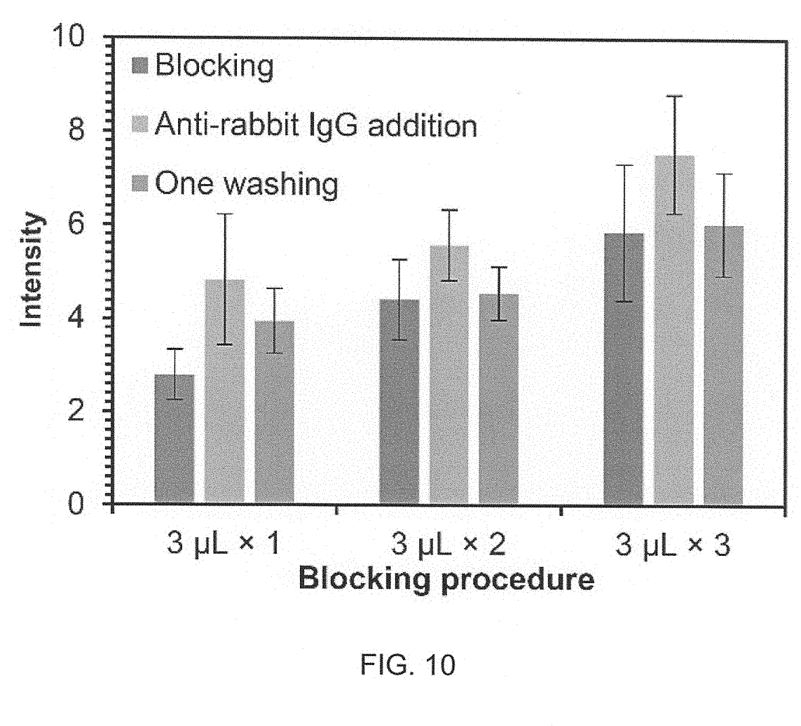

After confirming the success of protein immobilization after biofunctionalization, another experiment was undertaken to determine the blocking condition which is used to minimize the non-specific binding. The working electrodes 13 were first biofunctionalized, but no probe protein was immobilized, leaving GA as the ending groups to bind arbitrary proteins. The working electrodes 13 were blocked with 3 μL blocking reagent one or multiple times. FITC-tagged anti-rabbit IgG was then added to the working electrodes 13, and the working electrodes 13 were washed later. The fluorescence imaging results are shown in FIG. 10. With two and three additions of 3 μL of block reagent, the fluorescence intensity was virtually the same before addition of antibody and after washing, which means it is sufficient to minimize the non-specific binding. Since the probe proteins were not immobilized before blocking, the blocking reagent can be assumed to be enough to cover all the binding spots of GA on the surface 14.

### Electrochemical Characterizations

EIS biosensing is widely recognized as a sensitive, selective, and convenient biosensing mechanism. It can differentiate the phenomena on electrode surface from others in the electrochemical cell, and the obtained Nyquist plots could be interpreted by well-established electrical circuit models, to extract the information of interest. This mechanism requires no labelling molecules, whereas gold-standard ELISA always involves a labelling molecule, conjugated with the target molecule, for signal production or amplification.

In general, there are two categories of EIS processes: non-Faradaic process and Faradaic process. In experimentation, the Faradaic process was used, which involves electron transfer at the surface of the working electrodes 13. Faradaic-process-based biosensing typically utilizes the resistance of electron transfer (Ret) at the surface 14 of the working electrodes 13 as the sensing indicator, and it is considered highly sensitive and robust for detecting the insulation process of the working electrode surfaces 14 upon the binding of target molecules to the probe immobilized on the surfaces 14.

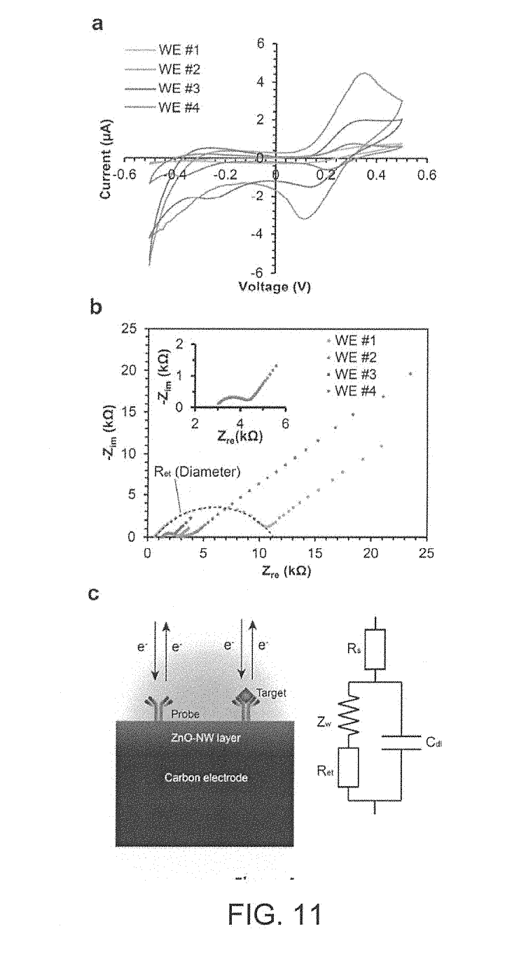

K₃[Fe(CN)₆]/K₄[Fe(CN)₆] was utilized as the redox pair to support the electron transfer. With this system, a series of cyclic voltammetry (CV) were run to found out the equilibrium point of the redox pair, which was then used as the direct current (DC) voltage in the following EIS measurements. The CV curves obtained with varied surface conditions of the working electrodes 13 through the process of biofunctionalization share similar equilibrium mid-point (FIG. 11a), and the DC voltage for EIS measurements was determined to be 245 mV (Table 1). The existence of ZnO nanowires was noted as largely increasing the current response, compared to the original carbon working electrodes 13, which could be attributed to the high electron mobility in ZnO nanowires. With the insulating linker molecules coming to the working electrode surface 14 in the process of biofunctionalization, the current response decreases. The insulating blocking reagent further brought down the current response.

TABLE 1
(For all the measurements, N = 5)

| | Redox mid-point (mV) |
|---|---|
| Working electrode condition | (average ± standard deviation) |
| Pristine carbon-ink electrode | 247.50 ± 11.18 |
| Pristine ZnO-nanowire electrode | 243.75 ± 11.59 |
| Biofunctionalized ZnO-nanowire electrode | 243.4 ± 7.31 |
| Biofunctionalized and blocked | 245.2 ± 9.66 |
| ZnO-nanowire electrode | |
| Biofunctionalized, blocked and | 244.60 ± 10.22 |
| stabilized ZnO-nanowire electrode | |

### EIS Measurements and Rabbit IgG Detection

The EIS measurements were performed on working electrode conditions varied through the biofunctionalization and blocking process, using 245 mV as the DC voltage and 5 mV as the AC voltage, in the frequency range of 100 k-0.1 Hz. The typical Nyquist plots for all the working electrode surface conditions, as shown in FIG. 11b, include a single “dome” shape followed by a slope at 45°. The Nyquist plot can be interpreted using an equivalent circuit model. The “dome” shape is represented by an electrical double-layer capacitor (C_{dl}) and an electron-transfer resistor (Rₑₜ) in parallel, the 45° slope implies the Warburg impedance (Z_{w}), and the resistance of the solution in the electrochemical cell is represented by Rₛ. In FIG. 11b, the Nyquist plots had different positions in the real axis of the impedance due to different values of the solution resistance (Rₑ), which was affected by the electrodes fabricated on the device, the fluidic condition in the reaction zone 16, and the composition of the fluid. Particularly with the immobilized and blocked working electrode (#4 in FIG. 11b), a long 45°-slope region was observed, which could be caused by the diffusion limitation on the working electrode surface once abundant blocking molecules are introduced to the working electrode surface. Because the long Warburg impedance range (corresponding to the 45° slope) does not provide information related to the biosensing process, the EIS frequency range was shortened to 20 k-20 Hz, for the functionalized and blocked working electrode, to get a close-up look at the Nyquist curves (inset in FIG. 11b). It is clear that the informational dome area is well-located within the 20 k-20 Hz range; thus, the frequency range of 20 k-20 Hz is applied in all the following EIS measurements. Based on the analysis, an electrical circuit is proposed to understand the EIS behavior of the device 10 (FIG. 11c). In the equivalent circuit model, Rₑₜ directly reflects the affinity-based binding of the capture probes and the target proteins, and thus is used as the readout signal of the EIS measurement. In the experiments described herein, the Ret value was extracted by measuring the diameter of the “dome” shape in Nyquist plots, using the commercial software of the potentiostat.

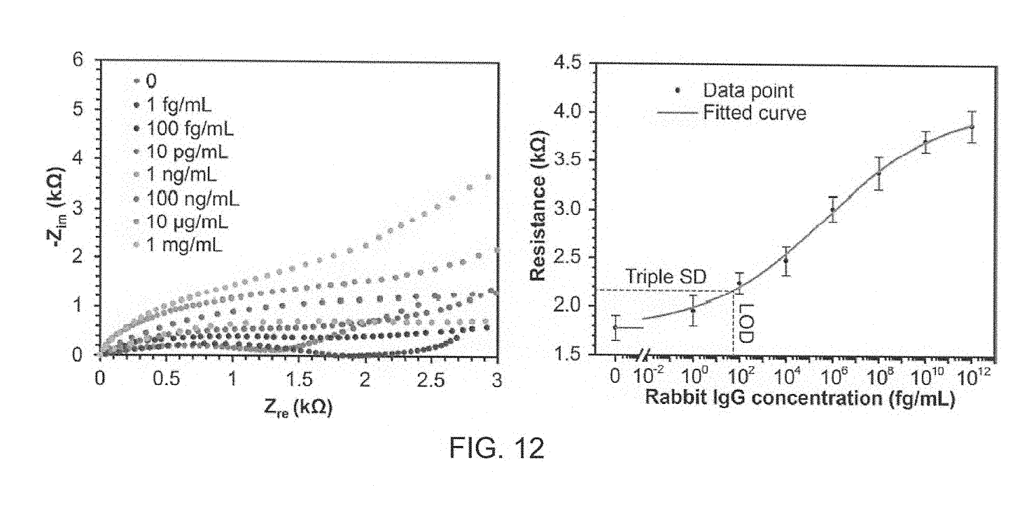

To quantify the effectiveness of the device 10, it was used for detecting rabbit IgG (as a model target) in phosphate-buffered saline (PBS) solution. The ZnO-nanowire working electrode 13 was functionalized with 25 μg/mL anti-rabbit-IgG solution. After adding the spiked PBS sample to the working electrodes 13, it was dried at room temperature for 15 min. As shown in FIG. 12a, the diameter of the dome (Ret) faithfully increases in response to increased rabbit-IgG concentration. The calibration results are fitted into S curve using the Hill equation (FIG. 12b):

$$y = 1.77512 + 2.28407 \frac{{x}^{0.17233}}{{( 4.96516 \times {10}^{5} )}^{0.17233} + {x}^{0.17233}} , {R}^{2} = 0.9964 , \qquad ( 1 )$$

The LOD was defined as the rabbit-IgG concentration that generates a readout signal three times the standard deviation (SD) of the signals from the zero-concentration samples. The LOD values was determined to be 60 fg/mL, which is at least 100-time lower than most of the previous paper-based immunosensors. The linear range was estimated to be 100 fg/mL-100 ng/mL.

### Detection of p24 Antigen in Human Serum

Human immunodeficiency virus (HIV) was selected as a target disease to demonstrate the practical use of the device 10. The HIV viral p24 antigen was used as the biomarker, and the p24 antigen detection is a part of the fourth-generation HIV diagnostic test. The ability to detect trace amounts of p24 antigens allows very early diagnosis of HIV infection, which could significantly increase the survival rate of patients and avoid additional health care costs due to late treatments. In the experiments, 10 μg/mL PBS solution of p24 antibody was used as probes on the ZnO-nanowire working electrodes. Commercial human serum samples, spiked with purified p24 antigen at different concentrations, were tested using the biosensor device 10 described herein. The present device design can also be readily extended to process whole blood samples, by integrating a blood separation membrane at the device inlet.

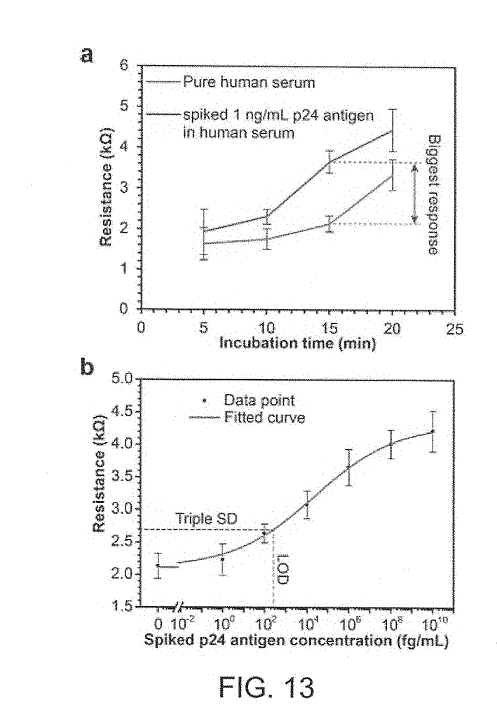

Considering the protein-rich environment of human serum samples, the incubation time was first tuned after sample addition. As shown in FIG. 13a, the Ret values measured on serum samples at zero and 1 ng/mL concentrations significantly increased after 20 min incubation, probably because the proteins and other chemical components in human serum attached firmly on ZnO nanowires after long-time evaporation at room temperature, and were hard to remove in the washing step. The deviation of Rₑₜ after 20 min incubation was also large, due to the protein residuals after working electrode washing. With incubation periods of 5 min and 10 min, on the other hand, the Rₑₜ of 1 ng/mL p24 antigen samples were measured to be small. It might be a result of incomplete capture within short incubations. 15 min incubation, among all the tested incubation times, generated the biggest response, and was thus used in the next experiments.

The calibration results of p24 antigen in human serum sample are organized in FIG. 13b. The data were fitted in S curve based on the Hill equation:

$$y = 2.11346 + 2.20386 \frac{{x}^{0.22458}}{{( 2.4858 \times {10}^{4} )}^{0.22458} + {x}^{0.22458}} , {R}^{2} = 0.99484 \qquad ( 2 )$$

The linear measurement range was estimated to be 100 fg/mL-1 ng/mL, with a LOD down to 300 fg/mL. With the ultralow LOD, and a wide linear measurement range, the device 10 showed the potential capability of early-stage clinical diagnosis. As a reference, the typical LOD of commercial ELISA p24 tests is 10-15 pg/mL.

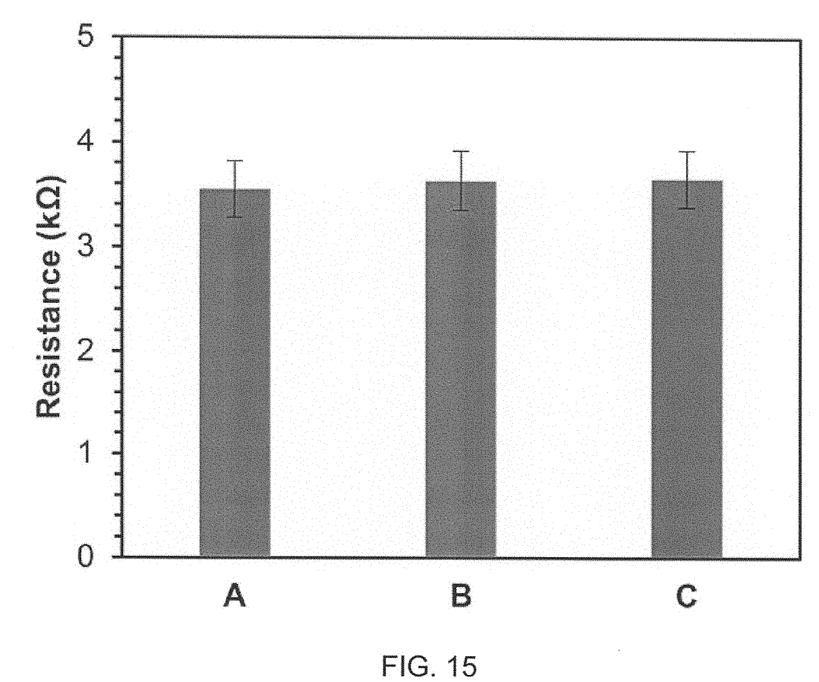

Experiments were also performed to examine cross-talk interference. Hepatitis C virus (HCV) was used as the interfering biomarker, because co-infection of HCV and HIV is often observed in clinic. The results (FIG. 15) demonstrated that the existence and varied concentration of HCV antigen does not interfere with the measurement of HIV p24 antigen.

### Investigation on Long-Term Storage

Device stability during storage is a concern in practical applications of biosensors, and the sensing performance of the device 10 was stored in eight weeks. There are two possible reasons that causes the degradation of the device's biosensing performance. First, the physical and/or chemical properties of ZnO nanowires may change over storage in dark. For instance, ZnO nanowires were found to become hydrophobic over long-time storage in dark, which will reduce the contact of the sample solution and the working electrode and thus the absorption of target proteins on the working electrode. Second, the conformation of the probe proteins immobilized on ZnO nanowire electrodes may change during storage.

Three groups of devices 10 were prepared and tested for p24 antigen detection. Group A was prepared as described above with blocking as the ending step on working electrodes, and was stored under the ambient laboratory environment. Group B was prepared the same way as group A, but as stored in sealed bags with desiccants. Working electrodes of Group C were treated with a commercial stabilizer (commonly used for immunoassays) after blocking, and the devices 10 were stored in sealed bags with desiccants. All the biosensor devices 10 were stored in dark at room temperature until the time of testing.

Regarding the concern of hydrophobicity of ZnO nanowires after long-term storage in dark, the surface 14 of the working electrodes 13 remained hydrophilic after storage. The ZnO nanowires grown on non-smooth and non-flat carbon-ink layer were not uniformly aligned, and researchers found that non-uniformed aligned ZnO nanowires may be less prone to becoming hydrophobic due to the random exposure of crystal planes. The biofunctionalization and blocking process introduced additional chemical groups and bonds to the ZnO nanowire surfaces, and the surface chemistry of ZnO nanowires were significantly altered.

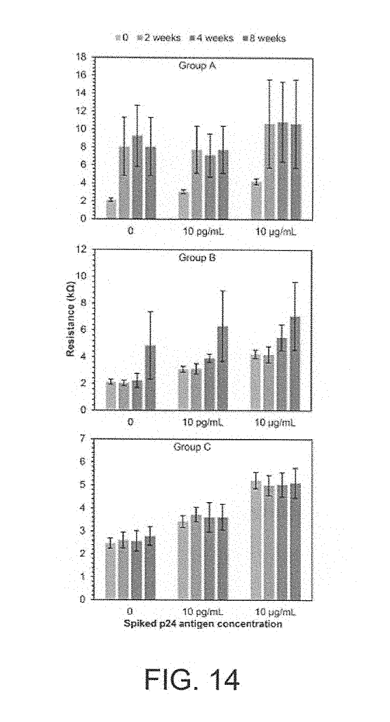

At time points of 0, 2, 4 and 8 weeks after device storage, human serum samples were tested, spiked with 0, 10 pg/mL and 10 μg/mL p24 antigen. The measurement results are shown in FIG. 14. Since the testing point at 2 weeks, Group A showed significantly increased Ret, and the standard deviations of the readout signals were augmented compared to those of the results at the 0-week testing point. In contrast, Group B and Group C had relatively stable performance over entire eight-week period. This implied that the low-humidity environment is a key factor to ensure the stability of the device 10. An increase of Ret seemed to appear after 4 weeks with Group B, while the devices 10 of group C always revealed stable biosensing performance during the eight-week storage. This implied that the commercial stabilizer also improved the device stability over long-term storage.

Testing of the Set 30 with Blood Samples

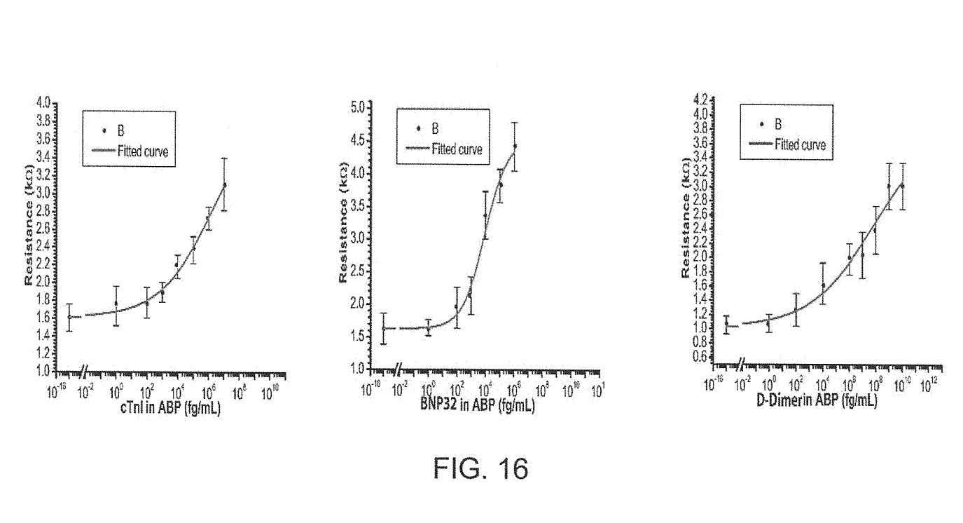

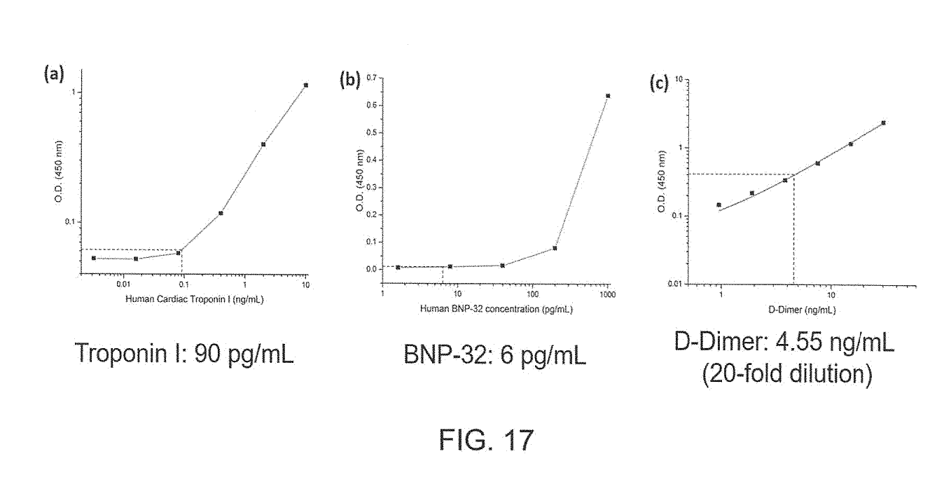

Three biomarkers were selected for testing: cardiac troponin I (cTnI), BNP32 and D-Dimer. cTnI and BNP32 are the biomarkers for cardiac diseases. They have different window time and specificity, and thus provide better accuracy and coverage of disease development. D-Dimer is the biomarker for blood clotting, and the purpose of detecting it is to evaluate the possibility of blood clots upon heart attack or angina and better analyze the condition of cardiac diseases on the patients. Calibration of the three biomarkers in artificial human plasma has been performed, which are the standard tests to quantify a biosensor's analytical performance. This calibration reveals the limit of detection (LOD) and the sensing range for these biomarkers. The calibration results of FIG. 16 imply that the biosensor device 10 covers the clinically relevant ranges of the three biomarkers, and the LODs (5 pg/ml for cTnI, 1 pg/ml for BNP32, and 2 pg/ml for D-dimer) are below the lower boundaries of the clinically relevant ranges, and are comparable to those of the standard ELISA tests (as shown in FIG. 17).

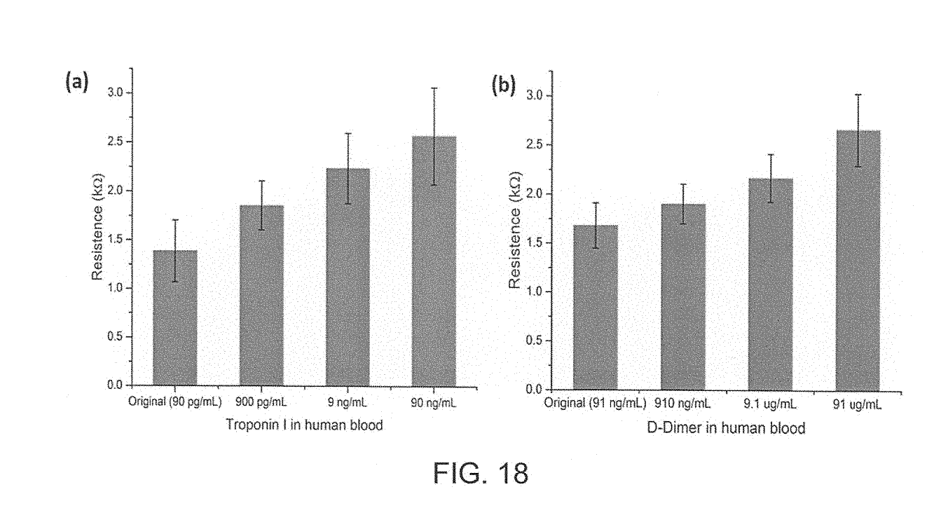

The calibration experiments of the devices 10 using spiked whole blood samples have been initiated. The calibration curves of cTnI and D-dimer, in their clinically relevant ranges, have been obtained (FIG. 18). These calibration curves will be used as the references (“look-up tables”) when patient sample tests are performed using the biosensors 10.

The device 10 described herein has potential to meet the requirements of point-of-care diagnostic testing, with features such as low device cost and pump-free liquid transportation. It has more advantages, including suitable biosensing performance, facile integration with nanomaterials, simplified and efficient operations, and a level of long-term stability. The on-chip integration of ZnO nanowires through hydrothermal growth process, the utilization of EIS sensing mechanism, and the efforts dedicated to study and improve long-term storage contributed to such achievements.

The integration of ZnO nanowires into the device 10 generated suitable biosensing performance. High surface-to-volume ratios of the ZnO nanowires contributed to the sensitivity, as more target biomarkers could be captured. Semiconductor ZnO nanowires also allowed the stable immobilization of probe proteins based on covalent bonding. The use of ZnO nanowires retained fairly simple device fabrication process without the need of clean-room fabrication, extreme experimental process, or significant costs. The improved biosensing capability with low costs is highly desirable in the applications of devices 10 to real-world diagnoses.

This device 10 involves label-free immunoassays and can differentiate different electrochemical phenomena in the sensing environment. The combination of these two features makes the device 10 suitably sensitive for protein detection and convenient to operate. A typical ELISA test on a 96-well plastic plate takes several hours or even days to obtain results and requires complex fluid manipulations and bulk/expensive equipment, whereas the device 10 provides ultrasensitive biosensing performance and much shorter assay time (25 min). All the operations on the device 10 can be conducted simply with a pipette and two reagent solutions (washing buffer and electron mediator). The EIS measurements require an impedance reader, for which a benchtop potentiostat was used in the above-described experiments. However, as the frequency of measurement interest lies in the range of 20 k-20 Hz as described above, cell-phone-based impedance measurement techniques (through the audio system of the cell phone, which has a frequency range of ≤20 kHz) are applicable for use by the device 10. The combination of the convenient device 10 and prevalent cell phones could would this advanced biosensing technology portable and more accessible, and will have significant impact on early-stage disease diagnosis at the POC.

The device stability over long-term storage is a critical parameter of any biosensors designed for practical uses. As explained above, the developed devices 10 were able to stably detect the target biomarker in human serum after eight-week storage. This was enabled by the low-humidity storage environment and the commercial stabilizer added to the working electrode to stabilize the protein probes. These results further prove the feasibility of using the device 10 for practical applications.

Encompassed herein, the devices 10 described herein can be used to detect a biomarker in a human sample such as blood, serum or a body fluid. Example of biomarkers encompassed herein are cardiac troponin I (cTnI), BNP32, D-Dimer, prostate specific antigen (PSA), prostate specific membrane antigen (PSMA), early prostate cancer antigen-1 (EPCA-1), early prostate cancer antigen-2 (EPCA-2), insulin-like growth factor binding protein 3 (IGFBP3), and any viral protein such as for example, but not limited to, p24 antigen. The device 10 can comprise at least one capture molecule, such as a probe, an antibody or an antigen specific for the targeted biomarker. The capture molecule can be coated on at least one electrode of the device prior to capturing the sample fluid as described herein, or can be present in the sample fluid which is captured on the growth nanomaterial surface.

The biosensing technology proposed herein, which includes enabling technical components such as direct integration of ZnO nanowires, probe immobilization based on covalent bonding, and EIS biosensing, will also have important applications beyond immunoassays. For example, probe deoxyribonucleic acids (DNAs) with modified ending groups can be immobilized on ZnO nanowires through a similar biofunctionalization process, and the device 10 could then be used for ultrasensitive DNA detection. Another embodiment is a miniaturized version of the device 10, with automated fabrication facilities. With smaller devices 10, especially with smaller working electrode 13, the sample consumption could be reduced, and better sensitivity might be expected.

The biosensor device 10 is a paper-based, sensitive immunobiosensor made possible by introducing to device in situ grown zinc oxide nanowires (ZnO nanowires) and a label-free electrochemical impedance spectrometry (EIS) biosensing mechanism. The growth of ZnO nanowires is directly performed on a carbon-ink electrode printed on paper substrate, without the concern of nanomaterial detachment or non-uniformity. The growth process is also convenient and low-cost. The existence of ZnO nanowires on device 10 enhances the sensing performance because of their high surface-to-volume ratio and support to stable covalent protein immobilization. The EIS biosensing mechanism has high specificity and sensitivity, as it differentiates the capacitance and resistance of different regions in the electrochemical cell. It requires no label, and thus simplifies the sensing operations. Using rabbit IgG in PBS, a limit of detection (LOD) of 60 fg/mL was demonstrated. The LOD for p24 antigen as an HIV biomarker is estimated to be 300 fg/mL in spiked human serum.

1. A biosensor device comprising:
a substrate base having a paper body, the substrate base supporting at least a first electrode having a growth nanomaterial surface on a portion thereof adapted to receive a sample fluid, and a contact end configured for connection to measuring equipment, and
a substrate cover having a paper body, the substrate cover defining a reaction zone in which the paper is exposed on both sides of the paper body, the substrate cover supporting at least a second electrode and at least a third electrode each having a connection end contacting the reaction zone and a contact end configured for connection to the measuring equipment,
wherein the reaction zone is superposed on the growth nanomaterial surface during use for wicking of the sample fluid through the reaction zone.

2. The biosensor device according to claim 1, wherein the substrate base and the substrate cover concurrently form a single-piece paper body, the substrate base and the substrate cover separated by a fold line for folding the single-piece paper body into superposing the reaction zone with the growth nanomaterial surface.

3. The biosensor device according to claim 1, comprising layering of hydrophobic material on the substrate cover surrounding and delimiting the reaction zone.

4. The biosensor device according to claim 2, wherein the second and the third electrode have a body printed onto the substrate cover.

5. The biosensor device according to claim 1, wherein the second electrode has a cane shape with an arcuate portion thereof being the connection end of the second electrode with the reaction zone.

6. The biosensor device according to claim 1, wherein the first electrode has an own paper body installable onto the substrate base.

7. The biosensor device according to claim 6, further comprising a layer of adhesive on the substrate base for securing the first electrode thereon.

8. The biosensor device according to claim 7, further comprising at least one backing strip on the layer of adhesive, the at least one backing strip defining a cutout contoured to receive the first electrode therein.

9. The biosensor device according to claim 6, wherein the paper body of the first electrode has a carbon layer extending from the contact end to the growth nanomaterial surface.

10. The biosensor device according to claim 9, wherein the paper body of the first electrode is layered with a hydrophobic material except on the growth nanomaterial surface.

11. The biosensor device according to claim 1, further comprising a substrate flap having paper body and a filter membrane superposed onto an exposed paper zone of the paper body, the exposed paper portion superposed onto the growth nanomaterial surface for wicking of a sample liquid filtered by the filter membrane, through the exposed paper zone, and onto the growth nanomaterial surface.

12. The biosensor device according to claim 11, wherein the substrate base and the substrate flap concurrently form a single-piece paper body, the substrate base and the substrate flap separated by a fold line for folding the single-piece paper body into superposing the exposed paper zone with the growth nanomaterial surface.

13. The biosensor device according to claim 1, further comprising a substrate flap having a washing element thereon, the substrate base and the substrate flap concurrently form a single-piece paper body, the substrate flap being separable from the substrate base for washing the substrate base.

14. The biosensor device according to claim 1, wherein the growth nanomaterial surface of the first electrode includes zinc oxide nanowires.

15. A method for preparing a biosensor device for an assay comprising:
exposing a growth nanomaterial surface of a first electrode to a sample fluid;
after capturing the sample fluid on the growth nanomaterial surface, applying a paper reaction zone in contact with a second and a third electrode against the growth nanomaterial surface of the first electrode to cause wicking action of the captured sample fluid through the paper reaction zone;
adding an electron mediator to the paper reaction zone; and
connecting the electrodes to measurement equipment to analyze the sample fluid.

16. The method according to claim 15, wherein applying the paper reaction zone against the growth nanomaterial surface comprises folding a first substrate portion against a second substrate portion.

17. The method according to claim 16, wherein folding a first substrate portion against a second substrate portion comprises adhering the first substrate portion to the second substrate portion.

18. The method according to claim 17, wherein adhering the first substrate portion to the second substrate portion comprises removing a backing strip from the first substrate portion to expose an adhesive.

19. The method according to claim 15, further comprising adhering the first electrode to a substrate base prior to applying the paper reaction zone against the growth nanomaterial surface.

20. The method according to claim 15, wherein exposing the growth nanomaterial surface of the first electrode to the sample fluid comprises deposing the sample fluid on a filter membrane, and wicking part of the sample fluid from the filter membrane through a paper substrate and onto the growth nanomaterial surface.
